(19) United States
(12) Patent Application Publication
(10) Pub. No.: US 2024/0390909 A1
(43) Pub. Date: Nov. 28, 2024

(54) DEVICE FOR COLLECTING LIQUIDS

(71) Applicant: SWISS SAFE COLLECT SA, NEUCHATEL (CH)

(72) Inventor: Benoit CAILLETEAU, BLONAY (CH)

(21) Appl. No.: 18/695,877

(22) PCT Filed: Sep. 20, 2022
(86) PCT No.: PCT/EP2022/076126

§ 371 Date: Mar. 27, 2024

(30) Foreign Application Priority Data
Sep. 28, 2021 (FR) FR2110230

(51) Int. Cl. B01L 3/00; G01N 1/10

(52) U.S. Cl. CPC B01L 3/505; B01L 3/5023; G01N 1/10; B01L 2200/16; B01L 2300/069; B01L 2300/123

(57) ABSTRACT

A device for collecting and analysing liquids is described. The device includes a flexible pouch comprising two walls configured to form an opening therebetween to introduce liquids, a reagent device that includes at least one element that is reactive with at least one component of a liquid, and a protective arrangement configured to protect the reagent device from contact with a jet of liquid coming from the opening to introduce the liquids.

### TECHNICAL FIELD

The present disclosure concerns a device for collecting liquids, for example biological liquids such as urine or vomit.

### BACKGROUND

Devices for collecting urine or vomit are known from the prior art, for example in European patent no. 3174512 having a pouch provided with a safety valve for substantially preventing the contents of the envelope from leaving via the opening.

These devices are, for example, used by bedridden patients, for example in hospital, or persons with low mobility.

### SUMMARY

The need to collect a patient's liquids is known, for example in order to reduce a risk of contamination by these liquids, and to ensure their disposal.

The advantage of analysing a patient's liquids in order to evaluate a pathology or the state of health of the patient is known, as a basis of medical biology.

Currently, a patient's liquids are transferred between different containers between their collection, their analysis and their disposal, which increases the risk of contamination linked to poor handling.

The problem therefore rises in designing a device that can ensure, in a safe and reliable manner, the functions of collecting liquids and easily disposing of them.

The present disclosure aims to respond to this problem.

The present disclosure relates to a device for collecting liquids, comprising:
a flexible pouch comprising walls configured to form an opening therebetween for introducing liquids,
a reactive device comprising at least one element that is reactive with at least one component of a liquid, and a protective arrangement configured to protect the reactive device from contact with a jet of liquid coming from the opening for introducing liquids.

The term “liquids” shall mean all types of liquids, for example biological liquids of a user, such as urine or vomit, or a mixture of biological liquids. The term “liquids” shall also mean non-biological liquids, for example aqueous-phase liquids such as wastewater or any other industrial liquid, or non-aqueous phase liquids, such as petroleum or oils.

The device for collecting liquids can thus have an application in medical or non-medical fields.

The term “element that is reactive with a component of a liquid” shall mean a device which reacts in characteristic manner in response to contact with a liquid and/or with chemical species contained in the liquid.

The term “flexible pouch” shall mean a pouch that can deform during filling with liquids. The use of a flexible pouch can make it possible, for example, to reduce the volume of the device for collecting liquids before use in order to facilitate its storage. However, it is understood that the pouch can also be a rigid or semi-rigid pouch.

The term “flexible” shall mean a material which can deform under its own weight. The term “semi-rigid” shall mean a material which deforms slightly under its own weight, but which can easily be deformed by hand. The term “rigid” shall mean a material which deforms slightly under its own weight and which is difficult to deform by hand.

Such a pouch can collect liquids while protecting the reactive device from an undesired contact with the liquids. The reactive device can then be placed in contact with the liquids collected so as to perform the reaction between the reactive element and the liquids.

Such a device thus enables a safe and reliable collection of liquids, while reducing the handling phases necessary for contact and reaction of the liquid with the reactive element.

In certain embodiments, the reactive device comprises a device for analysing liquids.

The device for analysing liquids is an element that is reactive with a component of a liquid, in the sense that it enables an analysis of the liquid to be formed following contact with this liquid. The term “analysis of liquids” shall mean detecting the presence of at least one component in the liquids and/or measurement of the concentration of at least one component in the liquids. The component may be a chemical species of biological origin, such as a protein, or of non-biological origin. The component can also be a cell, a biological tissue or a bacterium. The analysis results can be read by a user. Reading by a user can be performed visually, for example by comparison with a colorimetric scale or reading of an indicator, or by reading a signal, for example an electrical or digital signal.

With this configuration, the liquids can be collected in the flexible pouch without coming into contact with the device for analysing liquids, which makes it possible to avoid a premature contact of the device for analysing liquids with the liquids collected; such a premature contact risking degrading the quality of the measurements of the device for analysing liquids, or even preventing a measurement. In this way, the contact duration of the collected liquids with the device for analysing liquids can be controlled, so as to ensure the correct operation of the device for analysing liquids.

In particular, in the case where the collected liquids are the urine of a patient, the placing in controlled contact of the urine and the device for analysing liquids makes it possible to avoid contact with the urine from the start of micturition, which may be unsuitable for performing certain analyses.

In certain embodiments, the reactive device comprises an absorbent material.

The absorbent material is an element that is reactive with a component of a liquid, to the extent that it can absorb liquids, for example by retaining them in a solid phase or in a gel. The absorbent material can also flocculate the liquids.

Such a pouch enables the collection of liquids, then retaining them, for example in a solid phase or in a gel, and thus facilitating their disposal via appropriate waste management channels. In the case where the liquids are the urine of a patient following a medical treatment, for example chemotherapy, the absorption and disposal of the urine reduces the pollution of wastewater by toxic medical residues.

Moreover, the protection by the protective arrangement makes it possible to collect the liquids in the flexible pouch without it coming into contact with the absorbent material, which would lead to a premature absorption risking preventing an analysis of the liquids or harming the reliability of such an analysis.

In certain embodiments, the protective arrangement is configured to protect the device for analysing liquids from contact with the absorbent material.

This configuration can avoid any interference between the functions of collection and absorption of the liquids, which can then be ensured in a safe and reliable manner by the device.

The results of liquid analysis performed by the device of the present disclosure, could subsequently undergo an expert assessment by a health professional, who will be able to compare values measured by the device with reference values, for example the nominal values for a patient in good health, in order to perform a diagnosis.

In certain embodiments, the protective arrangement is configured to allow contact of liquids collected in the flexible pouch with the absorbent material and with the device for analysing liquids.

Once the liquids are collected, the contact with the absorbent material and with the device for analysing liquids makes it possible to respectively perform functions of absorption and analysis of the liquids.

In certain embodiments, the device comprises a non-return valve configured to obstruct a flow of liquids collected in the flexible pouch towards the outside of the flexible pouch.

This configuration can prevent the liquids from escaping from the flexible pouch. In particular, this configuration can allow handling of the pouch in order to place the liquids in contact with the absorbent material and with the device for analysing liquids, while preventing an accidental leak of liquids out of the flexible pouch.

In certain embodiments, the protective arrangement comprises at least one part of the non-return valve.

The non-return valve can thus have a double function of protecting the device for analysing liquids and the absorbent material from a jet of liquid coming from the opening for introducing liquids, and preventing an accidental leak of liquids outside of the flexible pouch. In this way, the structure of the device for collecting liquids is simplified, which further reduces the risks of failure of the device.

In certain embodiments, the non-return valve comprises a plurality of sheets.

In certain embodiments, the protective arrangement comprises a portion of flexible sheets which delimits a first chamber with a first wall of the flexible pouch and which delimits a second chamber with a second wall of the flexible pouch.

The first chamber and the second chamber can thus separately receive the collected liquids.

In certain embodiments, the device for analysing liquids is located in the first chamber and the absorbent material is located in the second chamber, the device for analysing liquids and the absorbent material being positioned in zones between the opening for introducing liquids and an end of the protective arrangement opposite the opening for introducing liquids.

Such a configuration makes it possible to distribute the liquids collected, between a first chamber in which the collected liquids can come into contact with the device for analysing liquids, and a second chamber in which the collected liquids can come into contact with the absorbent material. In this way, the functions of analysing the liquids and of absorbing the liquids can be performed without interfering with one another.

In certain embodiments, the device for analysing liquids is secured to one of the first wall and the portion of flexible sheets, and the absorbent material is secured to one of the second wall and the portion of flexible sheets.

Such a positioning can ensure the positioning of the device for analysing liquids and the absorbent material respectively, in the first chamber and in the second chamber, even in the case of handling of the collection device by a user.

In certain embodiments, the absorbent material is provided in a soluble envelope or is free in the second chamber or in the pouch.

The term “soluble” shall mean soluble in the liquids considered. In the case of aqueous-phase liquids, for example urine or vomit, soluble means soluble in water.

The soluble envelope can hold the absorbent material in position until it is in contact with the liquids, following which the dissolved soluble envelope can release the absorbent material in order to absorb the residual liquids of the pouch.

In certain embodiments, the absorbent material comprises sodium polyacrylate. Other absorbent materials can be provided, depending on the nature of the liquids to be absorbed, for example from super-absorbent polymers.

In certain embodiments, the device for analysing liquids comprises a reactive test strip.

A reactive test strip comprises reactive zones which react to the contact of liquids and make it possible to qualitatively or semi-quantitatively measure the presence of certain parameters of the liquids. A health professional will then be able to compare the values measured by the device with reference values, for example the nominal values for a patient in good health, and then perform a diagnosis.

In certain embodiments, the device for analysing liquids comprises a lab-on-chip. In certain embodiments, the lab-on-chip comprises a device for transmitting measurement data, which can then be collected and consulted by a member of medical personnel, who will then be able to be compare values measured by the device with reference values from a patient in good health, and then perform a diagnosis. These measurement results can also be used by a user other than a member of medical personnel in order to detect abnormal concentrations of values of interest, for example the concentration of creatine for athletes or nitrites and leukocytes for persons susceptible to urinary infections. A user can then make contact with a member of medical personnel who is able to perform a diagnosis.

The present disclosure also relates to a method for collecting liquids of a or by a user, inside a flexible pouch comprising walls configured to form an opening therebetween for introducing liquids, a reactive device comprising at least one element that is reactive to at least one component of a liquid, the method comprising the following steps:

collecting, in the flexible pouch, liquids of a or by a user through the opening for introducing liquids; protecting the reactive device from contact with a jet of liquid coming from the opening for introducing liquids.

In certain embodiments, the method comprises the following steps:

protecting an absorbent material of the reactive device from contact with a device for analysing liquids of the reactive device.

In certain embodiments, the method comprises the following steps:

hindering a flow of liquids collected in the flexible pouch towards the outside of the flexible pouch; turning over the flexible pouch so as to bathe the device for analysing liquids and the absorbent material in the liquids collected in the flexible pouch for a predetermined exposure duration.

The term bathe shall be understood to mean totally or partially immerse. For example, “to bathe a reactive device in the liquids”, shall be understood to mean to place at least the reactive element of the reactive device in contact with the liquids.

In certain embodiments, the method comprises the reading of a measurement of the device for analysing liquids and the absorption of liquids collected in the flexible pouch by the absorbent material.

### BRIEF DESCRIPTION OF THE DRAWINGS

FIGS. 1A to 1E show a same lateral view of a device for collecting liquids according to a first, second, third, fourth and fifth embodiment, respectively.

FIGS. 2A and 2B are schematic sectional views of the device for collecting liquids of the first and second embodiment.

FIGS. 2C and 2D are schematic sectional views of the device for collecting liquids of the third and fourth embodiment.

FIG. 3A is a schematic sectional view of a device for collecting liquids of the first embodiment in the turned over position after collection of the liquids.

FIG. 3B is a schematic sectional view of a device for collecting liquids of the third embodiment in the turned over position after collection of the liquids.

FIG. 4 is a schematic sectional view of a device for collecting liquids after collection of the liquids.

### DETAILED DESCRIPTION

The present disclosure will be described by referring to specific exemplary embodiments. However, it is obvious that modifications and changes can be made to these examples without going beyond the general scope of the present disclosure as defined by the claims. In particular, the individual features of different embodiments illustrated or mentioned can be combined in additional embodiments. Consequently, the description and the drawings should be considered as illustrating rather than limiting.

The present disclosure relates to a device for collecting liquids 100, and will be described in the case of a pouch comprising a safety valve comprising a plurality of sheets. A pouch comprising a safety valve comprising a plurality of sheets is known to a person skilled in the art, for example in European patent no. 3174512, the contents of which can be referred to during reading of the present disclosure, and will not be described in detail.

In the present disclosure, the device for collecting liquids 100 will be described in a direct orthogonal reference frame with axes X, Y, Z, wherein, by convention, the Z-axis is vertical. Indeed, when liquids are collected in the device, it is normally positioned such that its opening for introduction is directed upwards, so that the Z-axis is then vertical or in any event inclined with respect to the horizontal.

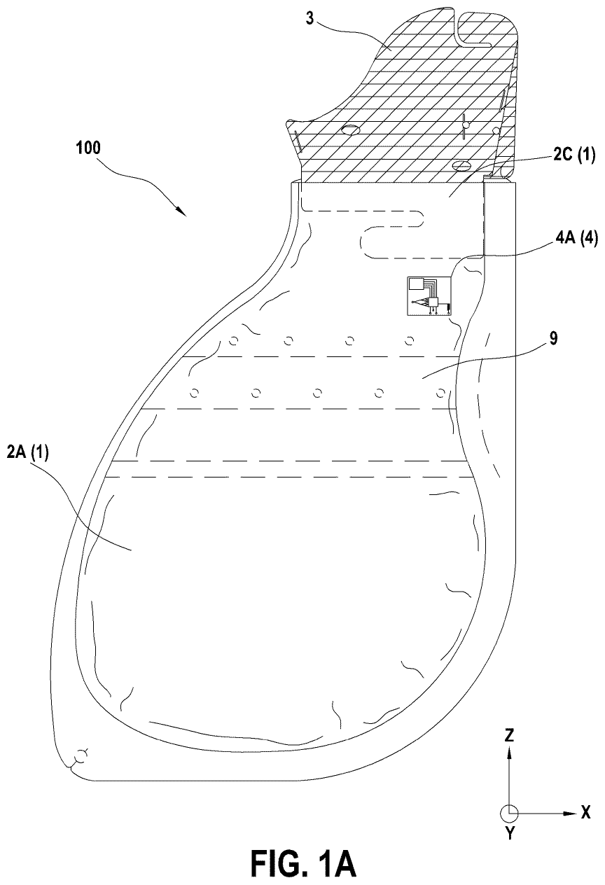

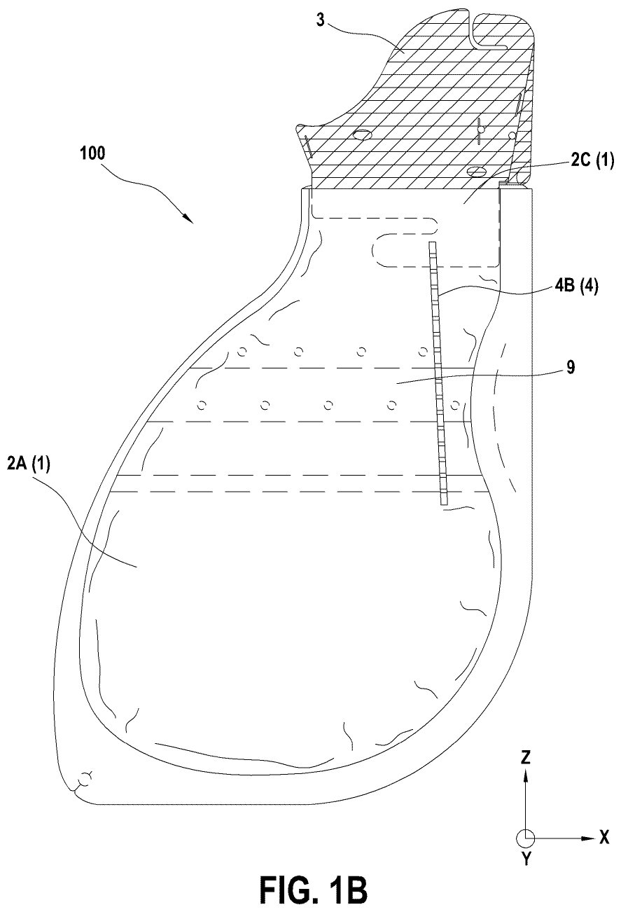

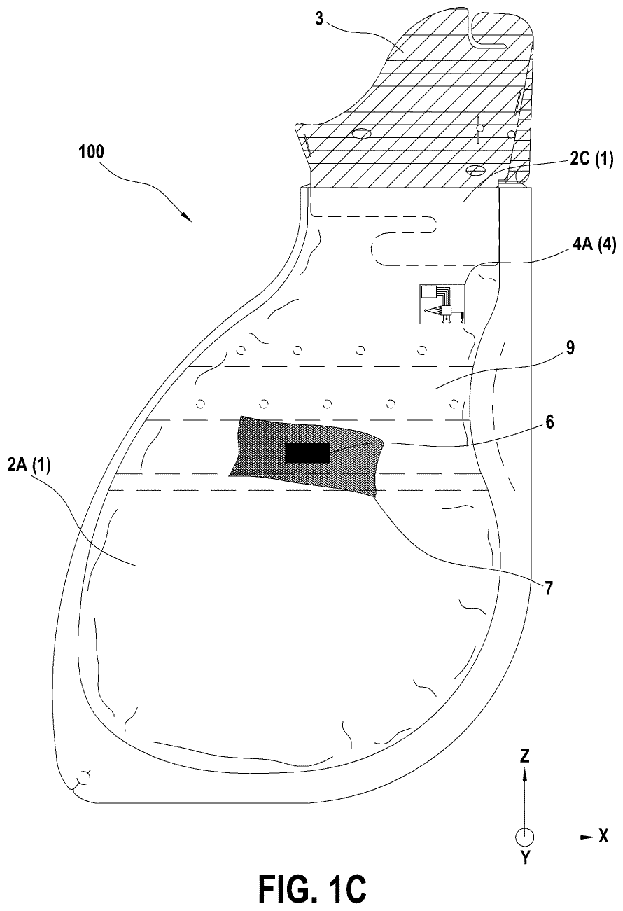

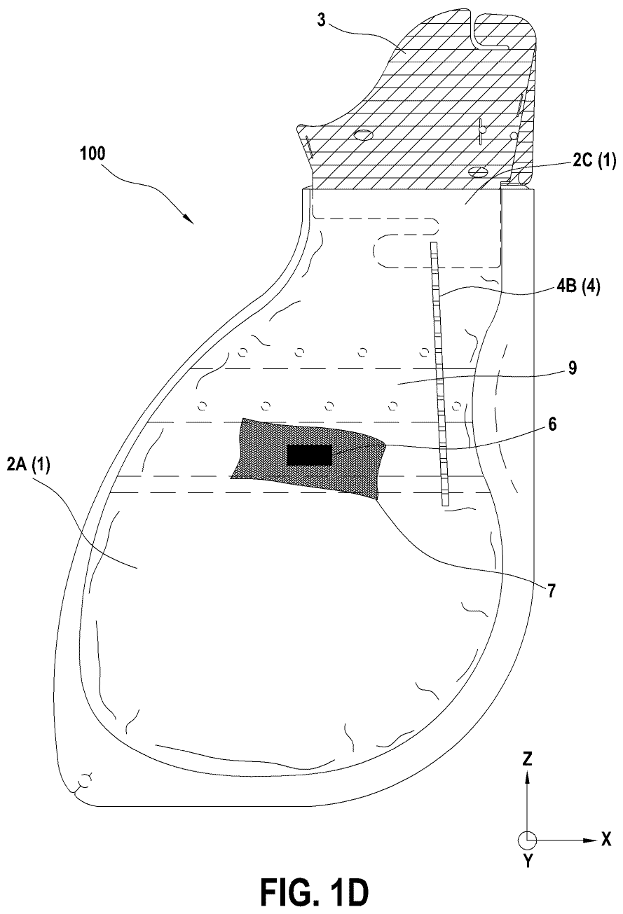

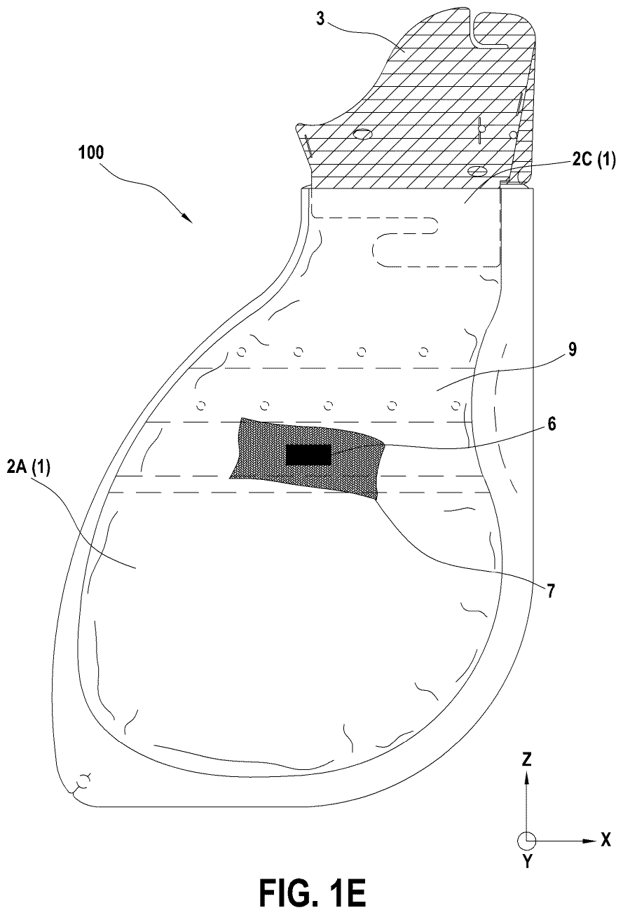

As shown in the view along the YZ plane of FIG. 1A, the device for collecting liquids 100 comprises a flexible pouch 1. The flexible pouch 1 is delimited by a first wall 2A and a second wall 2B, connected to one another over almost all of the contours of the walls 2A, 2B, for example by a weld. The free parts of the contours of walls 2A, 2B provide a neck 2C on which a sleeve 3 is secured.

The flexible pouch 1 can be made of a plastic material, for example polyethylene.

The device for collecting liquids 100 comprises a reactive device comprising an element that is reactive to at least one component of a liquid. For example, the reactive element is a device for analysing liquids 4, in the form of a lab-on-chip 4A.

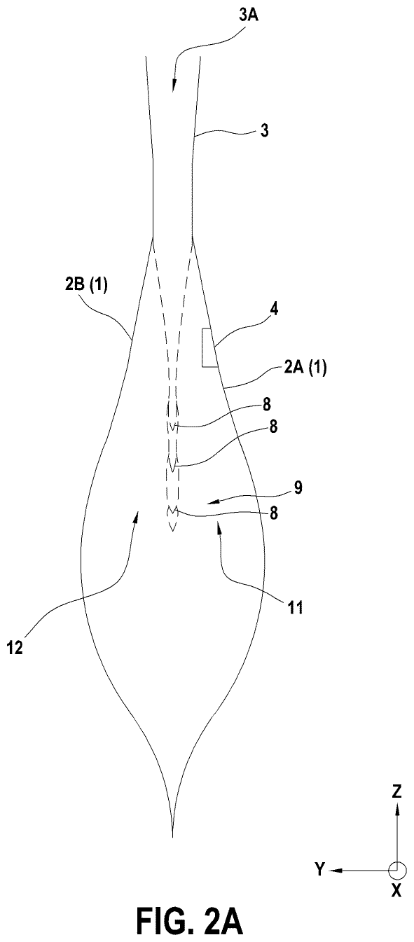

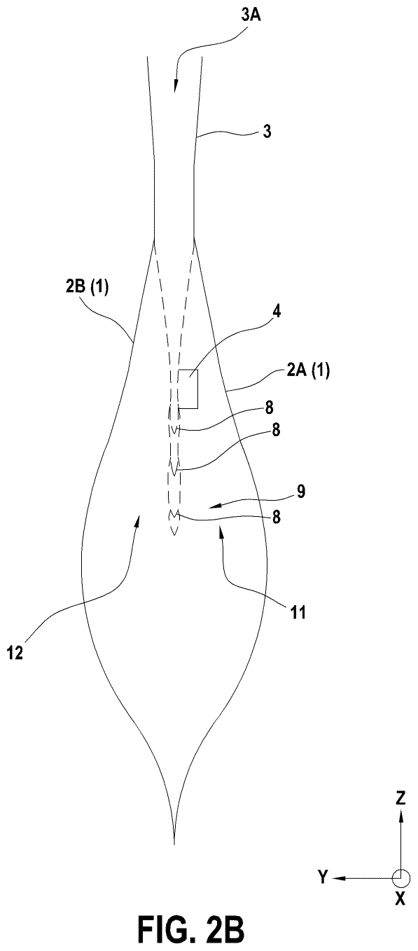

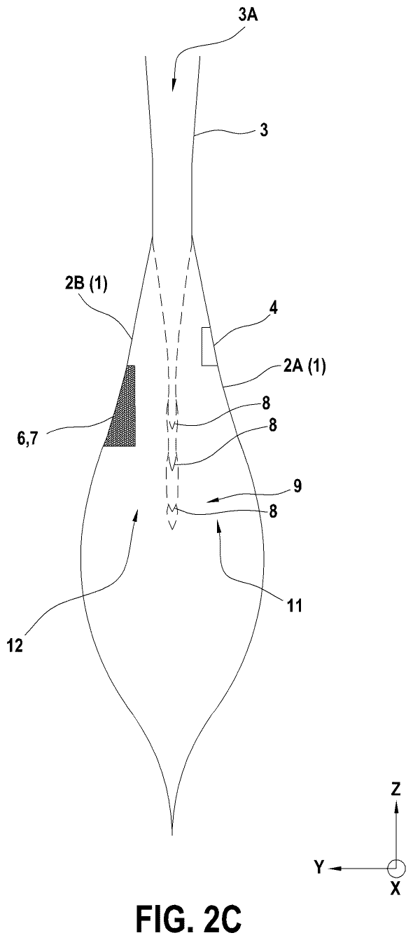

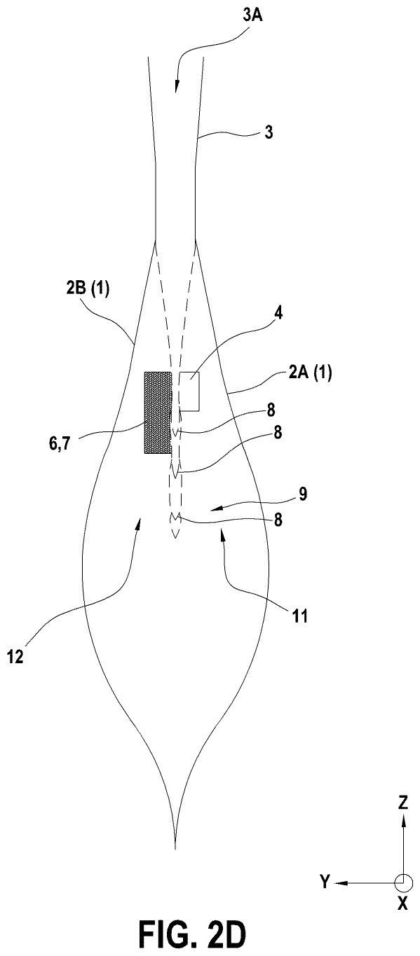

The device for collecting liquids 100 also comprises an absorbent material 6 and a non-return valve 9, which will be described in more detail in relation to FIGS. 2 to 4.

The embodiments of FIGS. 1A and 1B differ in that, in the embodiment of FIG. 1B, the device for analysing liquids 4 is a reactive test strip 4B. In general, the device for analysing liquids 4 can comprise a reactive test strip 4B and/or a lab-on-chip 4A.

A reactive test strip is known to a person skilled in the art, and is a strip provided with markers reacting to the presence or even to the concentration of certain chemical or biological markers, causing, for example, the appearance of a pattern or a change of colour.

A lab-on-chip is known to a person skilled in the art, and is a device for miniaturised biochemical measurement.

The embodiments of FIGS. 1C and 1D correspond respectively to the embodiments of FIGS. 1A and 1B, in which the reactive device further comprises a reactive element in the form of an absorbent material 6.

As shown in FIG. 1E, the reactive device can also comprise an absorbent material 6 and the device for analysing liquids 4 can be absent.

The positioning of the device for analysing liquids 4 and of the absorbent material 6 in the flexible pouch 1 will be described with reference to FIGS. 2A to 2D.

FIG. 2A shows a schematic sectional view of the device for collecting and analysing liquids 100 along the YZ plane, orthogonal to the XZ plane of FIG. 1A.

The sleeve 3 delimits an opening for introducing liquids 3A, via which liquids can enter into the device for collecting liquids 100.

A protective arrangement 9 is provided, in order to protect the reactive device from contact with a jet of liquid coming from the opening for introducing liquids 3A.

For example, the protective arrangement 9 protects the device for analysing liquids 4 and/or the absorbent material 6 from contact with a jet of liquid coming from the opening for introducing liquids 3A.

The protective arrangement 9 forms a flow passage for liquids in the extension of the opening for introducing liquids 3A.

The protective arrangement 9 is, for example, a non-return valve 9.

In particular, the non-return valve 9 is a non-return valve 9 comprising pairs of sheets 8 connected together by connection points provided so as to allow the passage of liquids coming from the opening for introducing liquids 3A to the inside of the flexible pouch 1, while preventing a flow of liquids from the inside of the flexible pouch 1 to the outside of the flexible pouch 1.

As shown in FIGS. 1A and 1E, the non-return valve 9 extends between two ends of the contour of walls 2A, 2B, so that the first wall 2A and the sheets 8 of the non-return valve 9 delimit a first chamber 11, and the second wall 2B and the sheets 8 of the non-return valve 9 delimit a second chamber 12, shown in FIG. 2A.

It is understood that the distinction between the first wall 2A and the second wall 2B, and between the first chamber 11 and the second chamber 12 is arbitrary, and that these are interchangeable.

The first chamber 11 and the second chamber 12 are separated by the sheets 8 of the non-return valve 9, and each form an open cavity which can communicate fluidically with each other beyond one end of the sheets 8 of the non-return valve 9.

It is understood that the extent of the first chamber 11 and of the second chamber 12 depends on the extent of the non-return valve 9. In particular, the first chamber 11 and the second chamber 12 are delimited by a plane perpendicular to the direction of flow of liquids from the opening for introducing liquids 3A and passing via an end of the sheets 8 of the non-return valve 9, in other words a plane perpendicular to the Z direction in the representation of FIG. 2A. In other words, the first chamber 11 and the second chamber 12 are between the opening for introducing liquids 3A and an end of the non-return valve 9 opposite the opening for introducing liquids 3A.

The first chamber 11 and the second chamber 12 thus correspond to positions inside the flexible pouch 1, which cannot receive liquids when collecting a jet of liquid from the opening for introducing liquids when the pouch is in the vertical position.

The analysis device 4 is provided in the first chamber 11. The analysis device 4 is, for example, secured to the first wall 2A, as shown in FIG. 2A, or secured to the sheets 8 delimiting the first chamber 11, as shown in the embodiment of FIG. 2B.

The embodiments of FIGS. 2C and 2D correspond respectively to the embodiments of FIGS. 2A and 2B, in which the device for collecting liquids 100 further comprises an absorbent material 6.

The absorbent material 6 is provided in the first chamber 11. The absorbent material 6 is for example secured to the second wall 2B, as shown in FIG. 2C, or secured to the sheets 8 delimiting the second chamber 12, as shown in FIG. 2D.

The protective arrangement 9 is provided to protect the device for analysing liquids 4 from contact with the absorbent material 6.

The absorbent material 6 can be provided in a soluble envelope 7. The soluble envelope is, for example, a water-soluble envelope 7, for example comprising water-soluble polymers such as polymers from the dextran and casein family.

The operation of the pouch will be described with reference to FIGS. 2A to 4.

After introducing liquids via the opening for introducing liquids 3A, the liquids flow and are collected in the flexible pouch 1 without coming into contact with either the device for analysing liquids 4, and, where applicable, with the absorbent material 6, protected by the non-return valve 9.

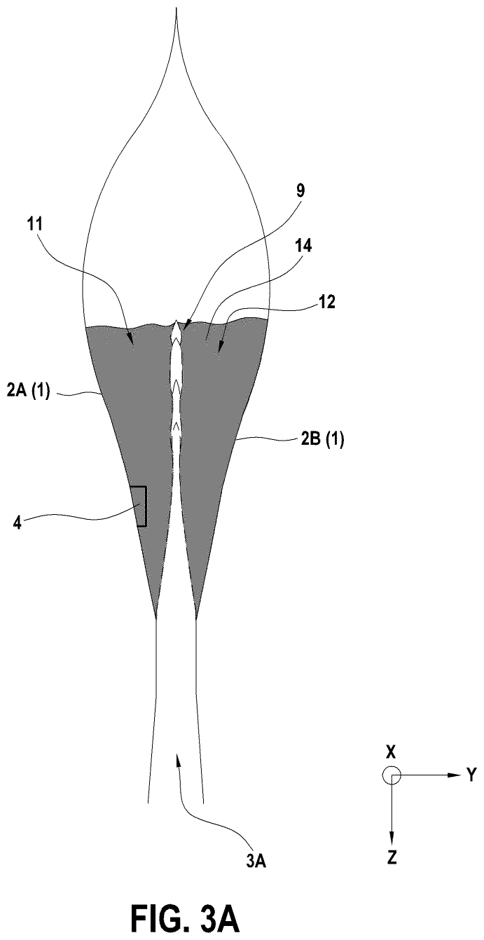

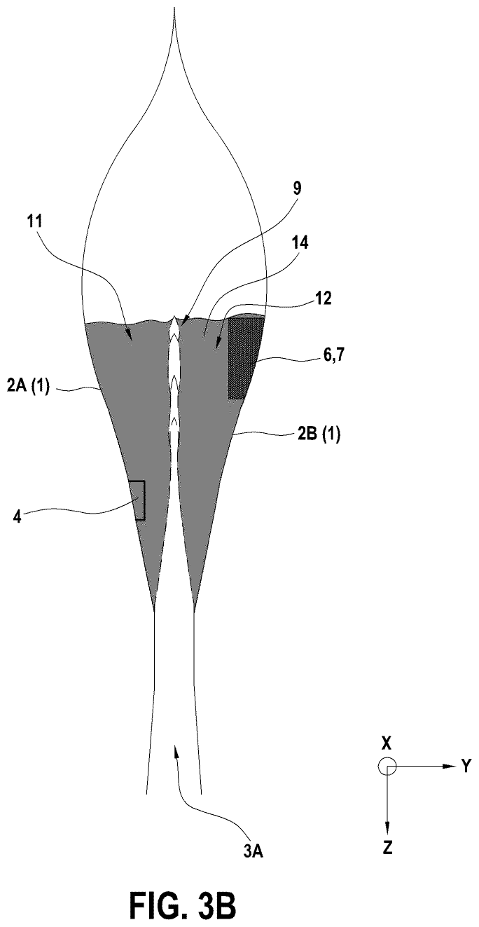

As shown in FIGS. 3A and 3B, the flexible pouch 1 can then be turned over so as to bathe the device for analysing liquids 4, and/or the absorbent material 6, in the liquids 14.

In particular, the liquids 14 fill the first chamber 11 so as to bathe the device for analysing liquid 4, and/or fill the second chamber 12, so as to bathe the absorbent material 6.

At the time of turning over the flexible pouch 1, the non-return valve 9 obstructs a leak of liquids 14 from the inside of the flexible pouch 1 to the outside of the flexible pouch 1.

The extent of the non-return valve 9 can be provided in a manner suitable for the volume of liquids normally collected in the pouch and thus received in the chambers 11 and 12 when the pouch is turned over. In particular, it can be designed so as to ensure a separation of the liquids 14 in the first chamber 11 and in the second chamber 12 without fluidic connection between the first chamber 11 and the second chamber 12, as shown in FIGS. 3A and 3B.

For example, in the case where the collected liquids are the urine of a user, the first chamber 11 and the second chamber 12 can each be provided to receive a volume greater than half the average volume of urine released during a micturition of urine.

It is understood, however, that in the turned over position of the flexible pouch 1, the level of liquids can also exceed the end of the non-return valve 9.

In the first chamber 11, the device for analysing liquids 4 bathes in the liquids 14 in order to perform an analysis measurement of liquids. In order to perform the analysis of the liquids, the duration of the bath in the liquids 14 must correspond to a predetermined exposure duration, which is a range of durations between a lower limit which is a minimum duration of contact enabling the reactions necessary for the measurement to take place, and an upper limit which is a maximum duration of contact beyond which the measurement is degraded, for example by dissolving of reagents from the device for analysing liquids 4 in the liquids 14. This predetermined exposure duration depends on the nature of the device for analysing liquids 4 and the markers used, and is, for example, on the order of ten to a hundred seconds.

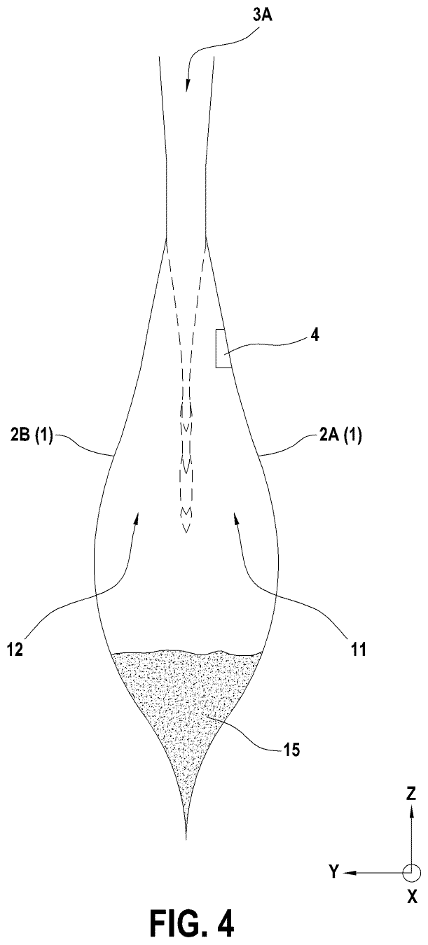

After a duration corresponding to the predetermined duration of exposure, the flexible pouch 1 can be turned over again so as to cause the liquids 14 and the residual absorbent material 6 to fall to the bottom of the flexible pouch 1, and the residual absorbent material 6 can then finish absorbing the liquids 14 so as to form a liquid residue 15, as shown in FIG. 4.

In the second chamber 12, the absorbent material 6 bathing in the liquids 14 absorbs part of the liquids 14. In the case where the absorbent material 6 is provided in a soluble envelope 7, the absorption of the liquids by the absorbent material 6 starts after release of the absorbent material 6 following the dissolving of the soluble envelope 7.

The quantity of absorbent material 6 is intended to be able to absorb all of the liquids 14 collected in the flexible pouch 1. For example, the absorbent material 6 may contain sodium polyacrylate, in quantities on the order of one to ten grams.

After a duration corresponding to the predetermined duration of exposure, the flexible pouch 1 can be turned over again so as to cause the liquids 14 and the residual absorbent material 6 to fall to the bottom of the flexible pouch 1, and the residual absorbent material 6 can then finish absorbing the liquids 14 so as to form an aggregate 15 of absorb liquids, as shown in FIG. 4.

The results of measurements of the device for analysing liquids 4 can then be read, by optical reading or by transmission via a wireless link to a server. These measurement results can then be used by a member of medical personnel, who will then be able to compare these measured values with nominal values of a patient in good health, and then perform a diagnosis. These measurement results can also be used by a user other than a member of medical personnel in order to detect abnormal concentrations of values of interest, for example the concentration of creatine for athletes or nitrites and leukocytes for persons susceptible to urinary infections. A user can then make contact with a medical professional who is able to perform a diagnosis.

In the case of a device for analysing liquids 4 requiring an optical reading, this is preferably performed through the flexible pouch 1, which must therefore be translucent or transparent.

The collecting of liquids from a user can be performed autonomously by this same user, or with the assistance of another user, for example a member of medical personnel.

The present disclosure has been described in the case of a flexible pouch 1 formed by the assembling of a first wall 2A and a second wall 2B. However, other structures of flexible pouches can be adopted. It is understood that in this case, the first wall and the second wall are distinguished by their position relative to the non-return valve 9, to the first chamber 11 and to the second chamber 12.

The present disclosure has been described in the case of a protective arrangement 9 comprising a non-return valve comprising sheets. However, it is understood that any type of non-return valve can be provided, for example depending on the nature of the liquids to be collected.

The present disclosure has been described in the case where the reactive device comprises a device for analysing liquid, and an absorbent material. It is understood that other elements that are reactive with at least one component of a liquid can be provided, and that each of the reactive elements can be provided independently of the others.

1. A device for collecting liquids, comprising:
a flexible pouch comprising walls configured to form an opening therebetween for introducing liquids,
a reactive device comprising at least one element that is reactive with at least one component of a liquid, and
a protective arrangement configured to protect the reactive device from contact with a jet of liquid coming from the opening for introducing liquids.

2. The device for collecting liquids according to claim 1, wherein the reactive device comprises a reactive test strip.

3. The device for collecting liquids according to claim 1, wherein the reactive device comprises an absorbent material.

4. The device for collecting liquids according to claim 1, wherein the reactive device comprises a reactive test strip and an absorbent material and wherein the protective arrangement is configured to protect the reactive test strip from contact with the absorbent material.

5. The device for collecting liquids according to claim 4, wherein the protective arrangement is configured to allow the contact of liquids collected in the flexible pouch with the absorbent material and with the reactive test strip.

6. The device for collecting liquids according to claim 1, comprising a non-return valve configured to obstruct a flow of liquids collected in the flexible pouch towards the outside of the flexible pouch.

7. The device for collecting liquids according to claim 6, wherein the protective arrangement comprises at least one part of the non-return valve.

8. The device for collecting liquids according to claim 6, wherein the non-return valve comprises a plurality of sheets.

9. The device for collecting liquids according to claim 1, wherein the protective arrangement comprises a portion of flexible sheets which delimits a first chamber with a first wall of the flexible pouch and which delimits a second chamber with a second wall of the flexible pouch.

10. The device for collecting liquids according to claim 9, wherein the reactive device comprises a reactive test strip located in the first chamber and an absorbent material is located in the second chamber, the reactive test strip and the absorbent material being positioned in zones between the opening for introducing liquids and an end of the protective arrangement opposite the opening for introducing liquids.

11. The device for collecting liquids according to claim 9, wherein the reactive device comprises a reactive test strip secured to one of the first wall and the portion of flexible sheets, and an absorbent material is secured to one of the second wall and the portion of flexible sheets.

12. The device for collecting liquids according to claim 1, wherein the reactive device comprises an absorbent material is provided in a soluble envelope.

13. The device for collecting liquids according to claim 3, wherein the absorbent material comprises sodium polyacrylate.

14. (canceled)

15. (canceled)

16. A method for collecting liquids of a or by a user, inside a flexible pouch comprising walls configured to form an opening therebetween for introducing liquids, a reactive device comprising at least one element that is reactive to at least one component of a liquid, the method comprising the following steps:
collecting, in the flexible pouch, liquids of a or by a user through the opening for introducing liquids; and
protecting the reactive device from contact with a jet of liquid coming from the opening for introducing liquids.

17. The method for collecting liquids of a or by a user according to claim 16, further comprising the following steps:
protecting an absorbent material of the reactive device from contact with a reactive test strip of the reactive device.

18. The method for collecting liquids of a or by a user according to claim 17, further comprising the following steps:
hindering a flow of liquids collected in the flexible pouch towards the outside of the flexible pouch; and
turning over the flexible pouch so as to bathe the reactive test strip and the absorbent material in the liquids collected in the flexible pouch for a predetermined exposure duration.

19. The method for collecting liquids of a user according to claim 18, comprising the reading of a measurement of the reactive test strip and the absorption of liquids collected in the flexible pouch by the absorbent material.
